(19) United States
(12) Patent Application Publication
(10) Pub. No.: US 2016/0074633 A1
(43) Pub. Date: Mar. 17, 2016

(54) Drug Coated Balloon Catheter and Method of Manufacture Thereof

(71) Applicant: Pine Medical Limited, Hong Kong (CN)

(72) Inventor: Silvio Schaffner, Hong Kong (CN)

(73) Assignee: Pine Medical Limited, Hong Kong (CN)

(21) Appl. No.: 14/845,821

(22) Filed: Sep. 4, 2015

(30) Foreign Application Priority Data
Sep. 11, 2014 (IT) PD2014A000233

(51) Int. Cl. A61M 25/10

(52) U.S. Cl. CPC A61M 25/10; A61M 25/1029; A61M 2025/1088; A61M 2025/1031; A61M 2025/105

(57) ABSTRACT

A drug coated balloon (DCB) catheter (4) has a connector (8), a shaft (20) extending from a proximal end (24) to a distal end (28) along an axial direction (X-X) and has a guidewire lumen (32) and an inflation lumen (36), the shaft being connected to the connector (8) on said proximal end (24), wherein the shaft (20) being provided with an inflatable balloon (48), fluidically connected with said inflation lumen (36) in order to be selectively inflated and/or deflated. Advantageously, the balloon (48) is coated with a drug to be delivered on a target lesion, an external wall (52) of the shaft (20), opposite to said lumens (32,36), is covered with a lubricant, and said guidewire lumen (32) is internally covered with a lubricant.

### CROSS-REFERENCE TO RELATED APPLICATIONS

This application claims the priority to Italian patent application No. PD2014A000233 filed Sep. 11, 2014, the entire disclosure of which is expressly incorporated herein by reference.

### BACKGROUND OF THE INVENTION

The present invention relates to a drug coated balloon catheter and the method of manufacture thereof.

In particular, it is known in the art to realize drug coated balloon (DCB) catheters comprising a shaft, extending from a tip (distal) to a connector (proximal), and a balloon which is expandable and which is covered or soaked with drug, in order to elute its drug directly on the target lesion.

Known Drug Coated Balloon Catheters (DCB catheters) are not lubricant coated. This leads to the fact that the DCB has higher friction in the interior wall of the target vessel or the interior wall of an introducer, sheath, guiding catheter or any other accessory used for intervention; and to the exterior wall of the guide wire used for intervention; therefore the catheter has problems to reach, or even cannot reach, the target lesion.

Sometimes the DCB catheter reaches the target lesion but, when moving into the vessel, it damages it because of said high friction: this damage can, for example, occur in correspondence of reductions of the lumen of the vessel or in correspondence of bifurcations of the vessel itself.

Moreover, the DCB catheters may encounter high friction and/or jamming between the internal wall of the shaft, defining the guidewire lumen, and the guidewire itself: this friction/sticking increases the problems of the balloon to reach the target lesion.

The problem of high friction between the guidewire lumen and the guidewire is increased in DCB catheters which have catheter shafts with coaxial or dual-lumen construction: one separate lumen is used so as to inflate the balloon. In fact, in such devices the guidewire lumen is reduced in diameter and then there is higher risk of blocking of the guidewire against the internal wall of the guidewire lumen.

Moreover, DCB catheters encounter higher friction than other types of balloon catheters due to the drug coating on the surface of the balloon; therefore the risk of jamming or grasping within blood vessel is really increased when using a DCB catheter with respect to other types of balloon catheters.

Therefore, it is not known in the state of the art a solution of DCB catheter which can easily reach the target lesion, without jamming against the internal wall of the vessel or against the guidewire.

### SUMMARY OF THE INVENTION

The purpose of the present invention is that of providing a catheter which overcomes the drawbacks mentioned with reference to the prior art; in other words a DCB catheter which can easily reach the target lesion without jamming and without damaging the internal wall of the cannulated vessel.

Such aim is reached by a catheter according to claim 1 and by a method according to claim 11.

Other embodiments of the catheter according to the invention are described in the subsequent claims.

### BRIEF DESCRIPTION OF THE DRAWINGS

Further characteristics and advantages of the present invention will be more clearly comprehensible from the description given below of its preferred and non-limiting embodiments, wherein:

FIG. 1 shows a perspective view of a drug coated balloon (DCB) catheter according to an embodiment of the present invention.

FIG. 2 shows a longitudinal section view of particular II of the catheter of FIG. 1.

FIG. 3 shows a longitudinal section view of particular III of the catheter of FIG. 1.

FIG. 4 shows a section view of the catheter of FIG. 1, taken along section line IV-IV shown in FIG. 1.

FIG. 5 shows a section view of the catheter of FIG. 1, taken along section line V-V shown in FIG. 1.

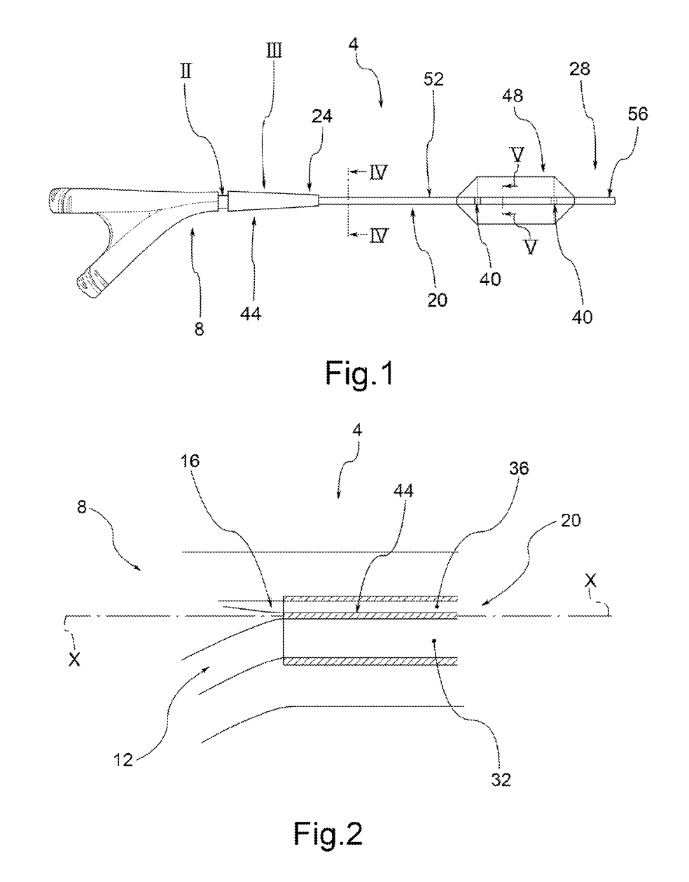

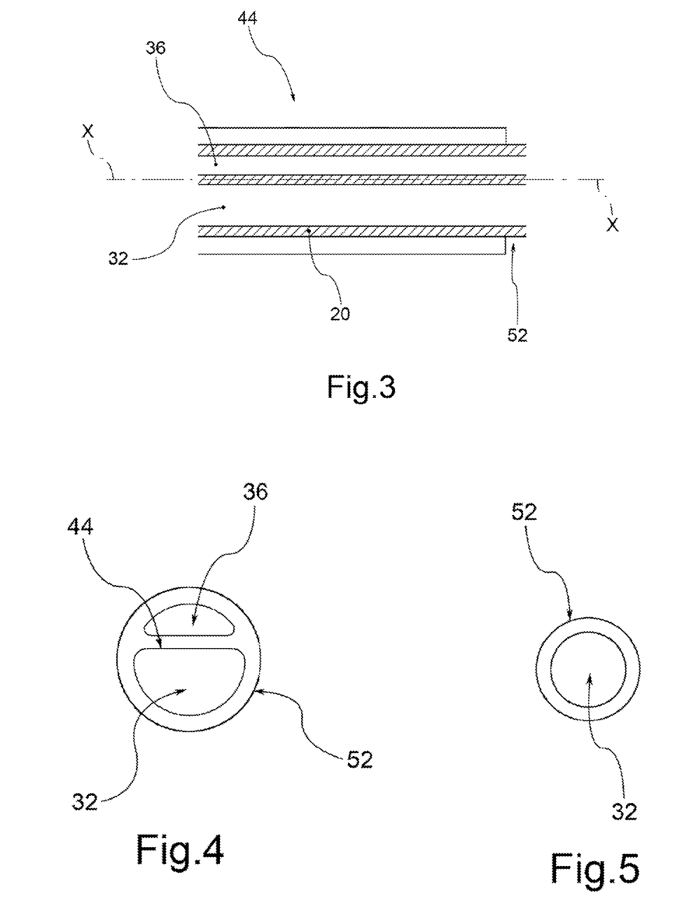

### DETAILED DESCRIPTION OF THE PREFERRED EMBODIMENTS

The elements or parts of elements common to the embodiments described below will be indicated using the same reference numerals.

With reference to the aforementioned figures, reference numeral 4 globally denotes a catheter, in particular, a drug coated balloon (DCB) catheter for releasing drug on a target lesion inside a blood vessel.

The catheter 4 comprises a connector 8 comprising a guide wire channel 12 and an inflation channel 16.

The guide wire channel 12 is useful for housing a guide wire for the guide and the insertion of the catheter 4 in a predetermined vessel.

Moreover, the inflation channel 16 is useful in order to send a pressure fluid to the distal end of the shaft of the catheter. For example, the pressure fluid may be gas or a liquid. Moreover, the inflation channel may be used as a perfusion channel, for example, of a contrast liquid.

The catheter 4 comprises a shaft 20 extending from a proximal end 24 to a distal end 28 along an axial direction X-X.

Moreover, the shaft 20 have a guide wire lumen 32 and an inflation lumen 36.

In particular, the catheter according to the present invention has at least a dual-lumen construction, comprising at least two lumens comprising a guidewire lumen 32 and an inflation lumen 36.

Preferably, said guidewire lumen 32 and inflation lumen 36 are not coaxial each other. For example, said guidewire lumen 32 and inflation lumen 36 are separated by an internal sept 44. For example said internal sept 44 delimits, at least partly, a wall of each of the separated lumens 32,36.

Anyway, the catheter 4 may have further lumens for different purposes.

The shaft 20 is connected to the connector 8 on said proximal end 24.

In particular, the shaft 20 is connected, at its proximal end 24, to the connector 8 so that said guide wire lumen 32 and inflation lumen 36 are mechanically and fluidically connected with said guide wire channel 12 and inflation channel 16 of the connector 8, respectively.

According to an embodiment of the present invention, the shaft 20 and the connector 8 are made up of a polymer material, such as Polyamide, Pebax, Polycarbonate and similar.

According to the invention, the shaft is provided with an inflatable balloon 48, which is fluidically connected with said inflation lumen 36 in order to be selectively inflated and/or deflated.

According to one embodiment, the balloon is provided to a couple of markers 40, positioned in correspondence of two ends of the balloon 48, defining the axial length of the balloon itself.

Advantageously, the balloon 48 is covered with a drug to be delivered on a target lesion.

The drug can be of any type, depending on the kind and positioning of lesion to be treated.

According to one embodiment, the drug formulation is a Paclitaxel solution.

According to the invention, an external wall 52 of the shaft 20, opposite to said lumens 32,36, is covered with a lubricant.

In this way, said external wall 52 which, during insertion of the catheter into the vessel, contacts the internal wall of the blood vessel or the internal wall of an introducer, sheath, guiding catheter or any other accessory used for intervention, is lubricated and can easily slip into the vessel itself.

According to the invention, the external wall 52 of the shaft 20 is covered with a lubricant except for said inflatable balloon 48.

In this way, the lubricant does not interfere with the action of the drug which has to be delivered on the target lesion.

The distal end 28 of the shaft 20 comprises a flexible tip 56 which aims the catheter to be cannulated inside blood vessels.

According to one embodiment, said tip 56 is covered with lubricant too.

Advantageously, said guidewire lumen 32 is internally covered with a lubricant.

Therefore, the shaft is provided with lubricant coating both externally, on the external wall 52, and internally, on the guidewire lumen 32.

According to one embodiment, said lubricant is a hydrophobic lubricant, like silicon or a hydrophilic lubricant.

According to a possible embodiment, the lubricant applied to the external wall 52 is a hydrophobic lubricant only, while the lubricant applied on the guidewire lumen 32 can be both hydrophilic and hydrophobic.

Hereafter the method of manufacturing a catheter according to the present invention will be described.

In particular the method of producing a catheter 4 according the invention comprises the steps of providing a shaft 20 extending from a proximal end 24 to a distal end 28 along an axial direction X-X, the shaft 20 having a guide wire lumen 32 and an inflation lumen 36.

The shaft 20 is provided with an inflatable balloon 48, fluidically connected with said inflation lumen 36 in order to be selectively inflated and/or deflated.

The method comprises the steps of:
coating the inflatable balloon 48 with a drug to be delivered on a target lesion,
coating with a lubricant the external wall 52 of the shaft 20, opposite to said lumens 32,36,
applying a lubricant into said guidewire lumen 32, in order to internally cover with a lubricant the guidewire lumen 32.

In particular, according to a possible and not limitative embodiment, the phase of applying lubricant into said guidewire lumen 36 comprises the steps of:
flushing, spraying or injecting the guidewire lumen 36 with a lubricating solution,
flushing the guidewire lumen 36 with compressed air to release coating in excess.

According to other possible embodiments, lubricant may be applied by plasma deposition.

According to an embodiment, the phase of applying lubricant into said guidewire lumen 32 comprises the steps of:
flushing the guidewire lumen 32 with injection of a lubricant coating solution,
coating only a part of the shaft 20 comprised between the balloon 48 and the connector 8.

Moreover, the phase of applying lubricant to the shaft 20 comprises the step of coating the external wall 52 of the shaft 20 by dipping it into a lubricant solution or by flushing or spraying it with a lubricant solution without influencing the balloon 48; also plasma deposition may be used in order to treat to the external wall 52 of the shaft 20.

As it can be seen from the description, the catheter and relative method of production according to the invention makes it possible to overcome the drawbacks mentioned with reference to the prior art.

In particular, it is possible to provide a DCB catheter which can reach the target lesion easily, without jamming and without damaging the cannulated vessel.

Therefore the use of the DCB catheter according to the present invention is advantageous because it is possible to reach the target lesion with lower friction and therefore less push, minimizing risk to damage the internal wall of the cannulated vessel also in the case of bifurcations or severe bending of the vessel itself.

Moreover, the DCB catheter may easily run along its guidewire without jamming: this is very useful for the user because he can ‘feel’ the geometry of the cannulated vessel, while pushing the DCB catheter throughout the vessel, without being confused by internal resistance of the catheter against the guidewire lumen. In this way the user can feel the geometry of the vessel and then can push forward or pull rearward the catheter basing on the resistance due to the anatomical geometry only, without being misled by internal friction and without risking to damage the internal wall of the cannulated vessel.

Of course, the lubricant used both internally and externally with respect to the catheter does not alter the functionality and the active principle of the drug of the balloon.

Moreover, it is possible to use DCB catheters, which have bigger and longer balloons than other types of balloon catheters, in order to treat any type of lesion or blood vessel, since the higher friction encountered by said DCB catheters is compensated and overcome by a more efficient lubrication of the catheter itself.

Therefore the risk of jamming or grasping within blood vessel is really decreased when using a DCB catheter according to the present invention.

Moreover, the use of lubricant coating according to the present invention is particular advantageous with a catheter having a dual lumen construction wherein said lumens are not coaxial each other. In fact, such a dual lumen geometry cannot be obtained by a multilayer extrusion with an inner material layer with lower friction than an outer material layer. Therefore, the use of a lubricant coating helps reducing friction significantly in a dual layer, not coaxial, catheter.

A person skilled in the art may make numerous modifications and variations to the catheters and methods described above so as to satisfy contingent and specific requirements, while remaining within the scope of protection of the invention as defined by the following claims.

What is claimed is:

1. A drug coated balloon (DCB) catheter (4), comprising
a connector (8),
a shaft (20) extending from a proximal end (24) to a distal end (28) along an axial direction (X-X) and having a guidewire lumen (32) and an inflation lumen (36), the shaft being connected to the connector (8) on said proximal end (24),
the catheter (4) having at least a dual-lumen construction provided with at least two lumens comprising said guidewire lumen (32) and said inflation lumen (36), wherein said lumens (32,36) are separated each other by an internal sept (44),
the shaft (20) being provided with an inflatable balloon (48), fluidically connected with said inflation lumen (36) in order to be selectively inflated and/or deflated,
characterised in that
the balloon (48) is covered with a drug to be delivered on a target lesion,
said guidewire lumen (32) is internally covered with a lubricant.

2. The drug coated balloon (DCB) catheter (4) according to claim 1, wherein an external wall (52) of the shaft (20), opposite to said lumens (32, 36), is covered with a lubricant.

3. The drug coated balloon (DCB) catheter (4) according to claim 1, wherein the entire external wall (52) of the shaft (20) is covered with a lubricant except for said balloon (48).

4. The drug coated balloon (DCB) catheter (4) according to claim 1, wherein the distal end (28) of the shaft (20) comprises a flexible tip (56), said tip (56) being covered with lubricant.

5. The drug coated balloon (DCB) catheter (4) according to claim 1, wherein said lubricant is a hydrophilic lubricant.

6. The drug coated balloon (DCB) catheter (4) according to claim 1, wherein said lubricant is a hydrophobic lubricant like silicon.

7. The drug coated balloon (DCB) catheter (4) according to claim 1, wherein the lubricant applied to an external wall (52) of the shaft (20) is a hydrophobic lubricant, and the lubricant applied on the guidewire lumen (32) is hydrophilic or hydrophobic.

8. The drug coated balloon (DCB) catheter (4) according to claim 1, wherein said connector (8) comprises at least a guide wire channel (12) and an inflation channel (16), the shaft (20) being connected, at its proximal end (24), to the connector (8) so that said guide wire lumen (32) and inflation lumen (36) are mechanically and fluidically connected with said guide wire channel (12) and inflation channel (16) respectively.

9. The drug coated balloon (DCB) catheter (4) according to claim 1, wherein the shaft (20) and the connector (8) are made up of a polymer material.

10. A method of producing a drug coated balloon catheter (4), comprising the steps of:
providing a shaft (20) extending from a proximal end (24) to a distal end (28) along an axial direction (X-X), the shaft (20) having a guide wire lumen (32) and an inflation lumen (36),
the shaft (20) being provided with an inflatable balloon (48), fluidically connected with said inflation lumen (36) in order to be selectively inflated,
the catheter (4) having at least a dual-lumen construction provided with at least two lumens comprising said guidewire lumen (32) and said inflation lumen (36), wherein said lumens (32,36) are separated each other by an internal sept (44),
characterised in that it comprises the steps of:
coating the balloon (48) with a drug to be delivered on a target lesion,
coating with a lubricant an external wall (52) of the shaft (20), opposite to said lumens (32,36),
applying a lubricant into said guidewire lumen (32), in order to internally cover with a lubricant the guidewire lumen (32).

11. The method of producing a drug coated balloon catheter (4) according to claim 10, wherein the phase of applying lubricant into said guidewire lumen (32) comprises the steps of:
flushing, spraying or injecting the guidewire lumen (32) with a lubricating solution, or using plasma deposition,
flushing the guidewire lumen (32) with compressed air to release coating in excess.

12. The method of producing a drug coated balloon catheter (4) according to claim 10, wherein the phase of applying lubricant into said guidewire lumen (32) comprises the steps of:
flushing the guidewire lumen (32) with injection of a lubricant coating solution,
coating only a part of the shaft (20) comprised between the balloon (48) and the connector (8).

13. The method of producing a drug coated balloon catheter (4) according to claim 10, wherein the phase of applying lubricant to the shaft (20) comprises the steps of:
coating the external wall (52) of the shaft (20) by dipping it into a lubricant solution or by spraying or flushing it with a lubricant solution without influencing the drug coated balloon (48).
